(19) United States
(12) Patent Application Publication
(10) Pub. No.: US 2026/0263127 A1
(43) Pub. Date: Sep. 10, 2026

(54) SCREW/HINGE ANTERIOR LIMB EPIPHYSIODESIS

(71) Applicant: Phoenix Children's Hospital, Inc., Phoenix, AZ (US)

(72) Inventor: Matthew A. Halanski, Scottsdale, AZ (US)

(21) Appl. No.: 19/558,159

(22) Filed: Mar. 5, 2026

Related U.S. Application Data
(60) Provisional application No. 63/767,313, filed on Mar. 5, 2025.

(51) Int. Cl. A61B 17/80; A61B 17/86

(52) U.S. Cl. CPC A61B 17/8066; A61B 17/8085; A61B 17/86

(57) ABSTRACT

The invention is generally a device and method of using the device that places a hinged plate over the anterior pubic symphysis and places screws across the anterior limbs of the triradiate cartilage that simultaneously guides growth to improve the coverage of the femoral head in a minimally invasive fashion without pelvic osteotomy.

### CROSS-REFERENCE TO RELATED APPLICATIONS

This application claims priority to and incorporates by reference U.S. Provisional Patent Application No. 63/767,313 filed on Mar. 5, 2025.

### FIELD

Generally, the present disclosure relates to an implant system that can include a plate and screw construct that is configured to modulate the direction of pelvic growth to improve acetabular (hip socket) positioning. More specifically, the present disclosure provides a system for correcting and improving the position of the hip socket to improve femoral head coverage to treat conditions such as hip dysplasia or instability with minimal morbidity.

### BACKGROUND

FIG. 1 illustrates how common children and adults around the world have various medical problems with their hips. In particular, children have hip dysplasia with high frequency. The most common cause of children having hip dysplasia is DDH or developmental dysplasia of the hip. Previously this was named congenital hip dyslasia (CDH). In addition, hip dysplasia is extremely common in neuromuscular conditions such as cerebral palsy, spinal muscular atrophy, and spina bifida. If non-operative measures are not successful at treating the disorder, large surgical procedures that cut and realign the proximal femur (osteotomy) and the acetabulum. (hip socket) to try increasing stability; depending on the level of success, total hip procedures may be needed.

Studies have been performed that demonstrate that these (osteotomy and hip reconstruction) procedures are among the most painful in all of orthopedics. They are associated with high morbidity and 4-6 weeks of non-weightbearing. As they are often bilateral in nature, this either means one large recovery with no weightbearing on either lower extremity for 1-2 months or two separate procedures and recoveries needing to be done.

### SUMMARY

This Summary section is neither intended to be, nor should be, construed as being representative of the full extent and scope of the present disclosure. Additional benefits, features and embodiments of the present disclosure are set forth in the attached figures and in the description hereinbelow, and as described by the claims. Accordingly, it should be understood that this Summary section may not contain all of the aspects and embodiments claimed herein.

Additionally, the disclosure herein is not meant to be limiting or restrictive in any manner. Moreover, the present disclosure is intended to provide an understanding to those of ordinary skill in the art of one or more representative embodiments supporting the claims. Thus, it is important that the claims be regarded as having a scope including constructions of various features of the present disclosure insofar as they do not depart from the scope of the methods and apparatuses consistent with the present disclosure (including the originally filed claims). Moreover, the present disclosure is intended to encompass and include obvious improvements and modifications of the present disclosure.

It is an object of the invention to guide growth of the pelvis and acetabulum to improve hip stability and coverage that will be useful in Developmental dysplasia of the hip in a minimal invasive manner.

It is an object of the invention to guide growth of the pelvis and acetabulum to improve hip stability and coverage that will be useful in Neuromuscular dysplasia of the hip in a minimally invasive manner.

It is an object of the invention to correct bilateral deformities using a single incision, a single implant, without cutting the bone.

It is an object of the invention to allow immediate weightbearing and minimal recovery.

It is an object of the invention to eliminate a painful recovery, allows immediate weightbearing, and can correct bilateral deformities with a single incision and implant.

An example embodiment of the invention is placed over the anterior symphysis pubis through a small incision. The hinge of the plate overlies the anterior pubic symphysis. Screws are then placed retrograde up the superior pubic rami into the ilium on both sides, crossing the anterior limb of the triradiate growth cartilage. By doing so the implant modulates growth of the pelvis and the acetabulum to bring the hip socket in a more forward facing position, improving the posterior, anterior, and lateral coverage of the femoral head. With greater acetabular coverage of the femoral head, stresses between the head and socket are more uniformly distributed. The invention may correct bilateral deformities using a single incision, a single implant, without cutting the bone. The invention may also allow immediate weightbearing and minimal recovery. Current devices only guide growth of the long bones.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the Drawings:

FIG. 1 depicts an illustration displaying information on how frequently the medical conditions occur that this invention, i.e. medical procedure, is intended to mitigate.

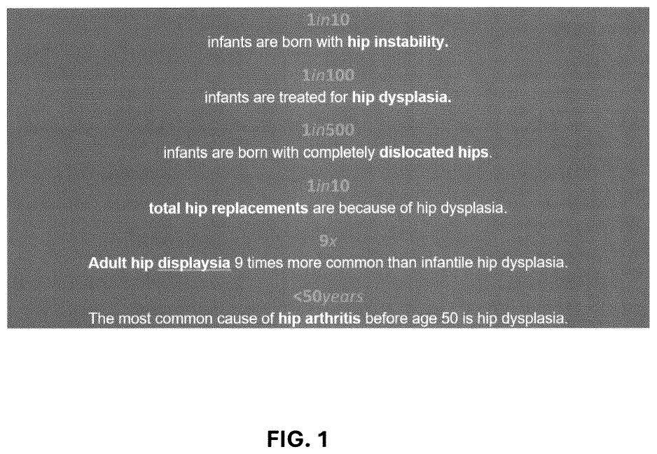

FIG. 2 depicts a small incision made and used during an embodiment of the invention.

FIG. 3 illustrates the positioning of the plate or hinge plate with the screws extending into the bone.

FIG. 4 illustrates the positioning of the plate or hinge plate with the screws extending into the bone.

FIG. 5 illustrates a control animal (gray on the left) and an experimental animal (white on the right) with a forward-facing position of the hip socket and the increased anterior and lateral coverage.

FIG. 6 is a flow chart depicting a method for installing the present implant device to modulate hip socket growth.

### DETAILED DESCRIPTION OF THE ILLUSTRATED EMBODIMENTS

The present invention generally relates to a medical procedure that uses a plate and screw construct, the Pega Medical/Orthopediatrics “Hinge Plate” system, to modulate the direction of pelvic growth to improve acetabular (hip socket) positioning. Using this system may correct and improve the position of the hip socket to improve femoral head (ball coverage) to treat hip dysplasia or instability with minimal morbidity.

In an embodiment of the invention, canulated screws may be used with the plate and the hinge may be placed across the pubic symphysis (rather than across a growth plate) and the screws may be placed so that they penetrate and cross the anterior limb of the triradiate cartilage anteriorly.

In the pelvis, acetabular structure and growth are complex. In particular, the pelvic ring consists of the sacrum and coccyx and the Os coxae, which consists of the ilium, ischium, and the pubis.

These tissues arise from the embryonic mesoderm and begin to take form on around the embryonic 28^{th} day as the limb bud forms. Chondrification of these structures eventually occurs between weeks 6-8 in utero and it is at this time that the three chondrification centers meet and fuse to form the acetabulum, more commonly referred to as the hip socket. The triradiate cartilage is formed at the intersection between the ilium, ischium, and pubis in the region of the acetabulum.

It is generally understood that the interstitial growth from expansion of the limbs of the triradiate cartilage act to increase the overall size, volume, and depth of the acetabulum during development. These limbs fuse around puberty, typically between 11-15 years in females and 14-17 years in males. Additionally, secondary ossification centers also form postnatally at the acetabulum rim around 9 years of age. The secondary ossification centers include the os acetabuli, located near the pubis that forms the anterior wall of the acetabulum, the epiphysis of the ilium forms the roof of the acetabulum (superiorly), while the secondary center of the ischium forms the posterior acetabular wall. Together the growth of these secondary centers expands outwards contributing to the overall depth of the acetabulum.

These centers enlarge and also fuse later in adolescence.

At birth, the majority of the articular surface is located posteriorly. Much of the acetabular shape and depth is established by eight years of age.

An opportunity exists to better define the relationship between the complex acetabular shape and its multiple growth centers. Sufficient pelvic growth is available during childhood to employ growth modulation strategies aimed at redirecting posterior acetabular articular cartilage into a more desirable location. The present disclosure provides an implant system and method that is configured to provide such growth modulation to enhance and control formation of the acetabulum via interactions with the various growth plates of the triradiate cartilage.

In general, longitudinal bone growth can be modulated, potentially eliminating the need for invasive procedures.

“Guided growth” or “growth modulation” is a therapeutic strategy that has been used in pediatric orthopedics. The strategy relies on asymmetric restriction of growth at a physis (growth plate) to achieve a desired modulation of that bone growth. Using this minimally invasive technique, angular deformities can be corrected over time by modulating future growth.

Bone growth modulation is a relatively low morbid approach that, if available, has largely replaced the use of much more invasive osteotomies for limb deformities. While permanent epiphysiodeses (ablation of the growth plate) have been described, reversible hemi-epiphysiodesis is more commonly employed to treat angular deformities to allow the proper “dose” of correction to be administered without over-correcting the deformity. Similar strategic restriction of pelvic growth centers may allow minimally invasive, osteotomy-free, acetabular redirection.

Pelvic growth modulation can therapeutically re-orient the acetabulum. In one approach, thermal symphysiodesis is an ablative veterinary procedure that re-orientates the acetabulum in skeletally immature canines. Using this approach, thermal destruction of the anterior, midline, pubic symphysis in young dogs caused premature pubic bone fusion. With subsequent posterior growth, the acetabular version (direction) can be shifted to increase posterior coverage allowing the walls of the acetabulum to extend further anteriorly and laterally.

In contrast to the thermally destructive approach, the present disclosure provides an implant device that can achieve similar results, while being significantly less destructive and titratable. In combination with the present implant system, a procedure, termed SHALE for Screw Hinge Anterior Limb Epiphysiodesis, is configured to modulate pelvic growth with particular focus on the development of the acetabulum. In applications, the present approach can achieve angular corrections equal or greater than that reported for triple Innominate osteotomy.

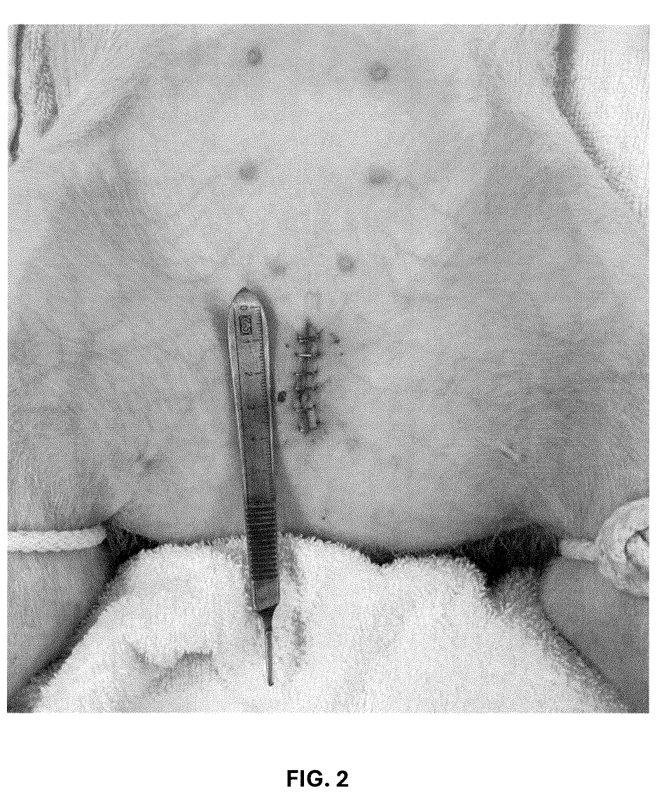

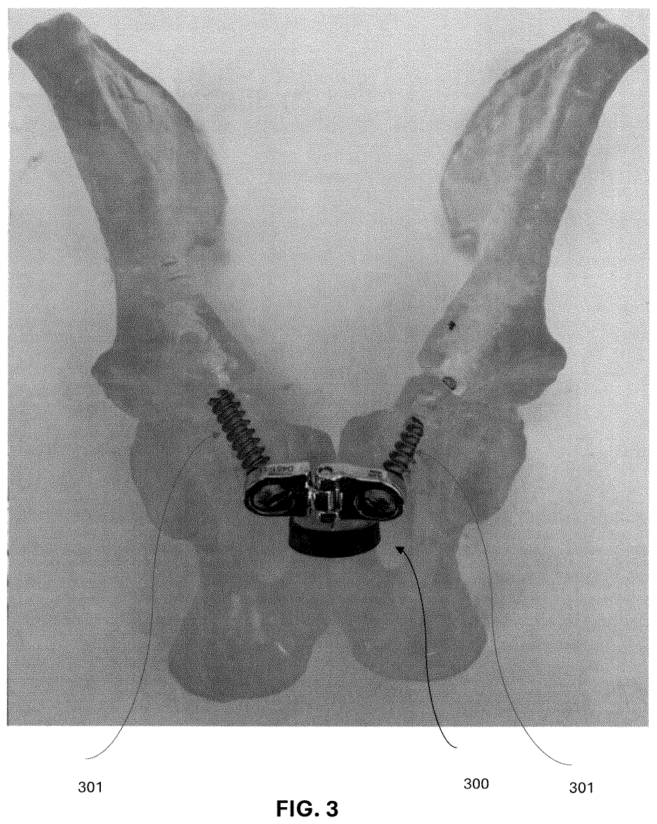

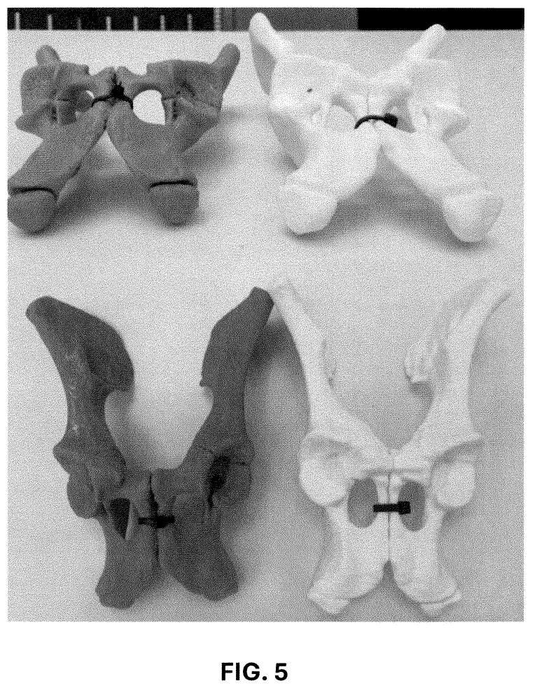

With reference to FIGS. 2-5, a procedure on an animal using the invention will now be described. During induction the animal was placed in a prone position. Following this the animal was placed in a supine position with hind limbs gentle held flexed and abducted with ties. The anterior symphyseal/groin region was clipped and scrubbed with Prodine scrub, then 70% Isopropyl alcohol, alternating three times. It was scrubbed with 50/50 chlorhexidine, followed by 70% Isopropyl alcohol, and finally sprayed with 70% Isopropyl alcohol. The region of interest was draped. A 4-cm longitudinal incision was made in the adduction crease over the pubic symphysis. Blunt soft tissue dissection, separating the muscle from the midline raphe about 1 cm on each side. The pubis was then palpated and muscle swept off the ventral surface of the pubis. Scissors were taken to cut the central raphe down onto the pubis, and elevators used to tease the tissue off the pubic symphysis. Dissection was performed to expose the top of the pubis as well as the inferior margin allowing Homan type retractors to be placed into the right and left obturator foramen and over the top of the pubis. Additional dissection was performed based on the style of implant used.

This animal underwent the modified symphyseal hinge plating with bilateral anterior limb screw epiphysiodesis. Dissection along the superior and inferior margin of the superior rami was performed out to the anterior edge of the hip joint. An awl was used to create a hole in the anterior cortex of the rami just medial to the superior aspect of the hip joint. A Lenke style spine awl was then used to create a path for the screw 301 medial to the hip joint, lateral and distal to the inner table and pelvic brim, that crossed the anterior limb of the triradiate cartilage. This was done under fluoroscopy, Steinmann pins were placed in these tracks and confirmed under fluoroscopy. The center of the two-holed plate 300 (Hinge plate Pega Medical/Orthopediatrics, Warsaw IN) was positioned over the symphysis bridging the symphysis anteriorly and the canulated screw 301 placed over the Steinmann pins crossing the triradiate cartilage under fluoroscopy. While out of the joint on fluoroscopy, the trajectory of these screws 301 were close, therefore they will be watched carefully post-op.

The incisions were irrigated with cephazolin in sterile saline and allowed to soak for 4 minutes. Then the incisions were irrigated with sterile saline and closed. The fascia was closed using 0-vicryl in a Ford interlocking suture pattern, 2-0 vicryl was used to close the subcuticular layer (simple interrupted pattern). Skin was stapled using stainless steel 35W staples. Incisions were instilled with bupivacaine. The animal was then recovered.

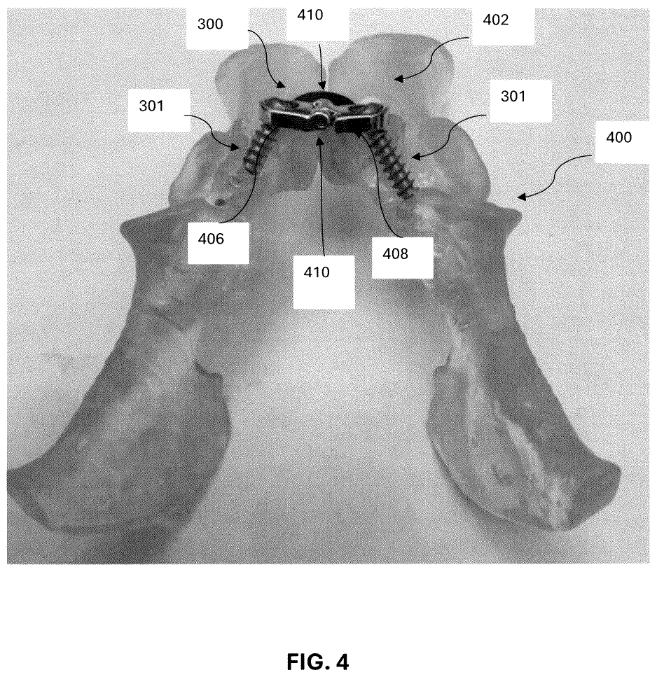

An example installation of the present system is depicted in FIG. 4, which shows the present implant system 402 installed into an example pelvis 400. As illustrated, implant system 402 includes a central hinge plate 404. Central hinge plate 404 includes a first plate portion 406 and a second plate portion 408, which are coupled at hinge 410. Central hinge plate 404 may be constructed using any suitable materials, including metals, such as surgical steel or titanium. Alternatively, central hinge plate 404 could include other biocompatible materials, such as certain polymers, resins, or other materials. Central hinge plate 404 may be a manufactured component, or could be 3D printed, depending upon the particular use case.

Each individual plate, first plate portion 406 and second plate portion 408 of central hinge plate 404, includes a hole or opening configured to receive a surgical screw. These screw holes may be formed directly through each of central hinge plate 404 and first plate portion 406 (e.g., at an angle orthogonal to a surface of each of first plate portion 406 and second plate portion 408) of may be formed at angle to such a surface. The two screw holes in each plate may be formed at the same angle (e.g., same offset angle) or may be formed at different angles, again, depending upon the application.

Although in an embodiment, central hinge plate 404 is described as comprising two distinct plates that are coupled at a hinge connection, it is important to understand that other configurations of central hinge plate 404 could be used. For example, instead of two plate connected at a hinge, the two plates may be connected by a flexible member that comprises a flexible yet sufficiently robust biocompatible material. In that case, central hinge plate 404 may comprise two distinct plates coupled by the flexible material. Or, alternatively, central hinge plate 404 may comprises a single component that provides the functionality of the two plates and the hinge member, such as by being formed of sufficiently flexible material.

Alternatively, central hinge plate 404 may be comprise a single fixed component that does not include a hinge. In that case, central hinge plate 404 may be made of a single piece of material that is shaped to mimic the shape of central hinge plate 404 as depicted in FIG. 4, but where the position of the plates is fixed to one another by a central fixed member. In such an embodiment, because the flexibility of central hinge plate 404 is significantly reduced as compared to other embodiments of central hinge plate 404 that include a hinge or other flexible coupling material between the two plates, the screw holes may be designed such that the screw holes are configured to retain screws, but while allowing for some movement of the screw within the screw holes. In that case, the allowed movement of the screws with respect to the fixed embodiment of central hinge plate 404 could allow for some flexibility and movement of the overall present implant system 402.

For example, in an embodiment, plate 404 may include polyetheretherketone. In that case, plate 404 may comprise a 3D printed component custom-printed for a particular patient. Alterantively, plate 404 may include a biocompatible polymer or biocompatible resign.

To install present implant system 402, an incision is made over the patient's pubic symphysis to expose the ventral surface of the pubis. Central hinge plate 404 is then positioned over or proximate (e.g., within 5 cm of) the pubic symphysis.

On a first side of the pelvis a hole is then formed in the anterior cortex of the rami just medial to the superior aspect of the hip joint. A first screw 301 can then be inserted through a first screw hole of central hinge plate 404 and placed into the hole. The screw is then threaded into the hole such that the screw penetrates through the anterior limb of the tridate cartilage of the pelvis in a first hip socket of the pelvis. A similar process is used to place a second screw through the second screw hole of central hinge plate 404 and into the pelvis towards a second hip socket of the pelvis.

In this configuration, hinge 410 of central hinge plate 404 allows for some flexibility at the pubic symphysis. The two screws 301, in combination with central hinge plate 404 form a structure that, as the pelvis grows, applies a tension force to the tridate cartilage and, particularly, the anterior limb thereof, which generates a modulating force to the tridate cartilage thereby controlling growth of the pelvis and, particularly, the acetabulum of the pelvis.

In alternative placements of present implant system 402, such as where a patient only requires growth plate modulation on one side, the screw on the side requiring modulation may be installed so as to pass through the tridate cartilage, which results in the desired growth plate modulation on that side. However, for the side that does not require such modulation the screw may be installed (e.g., via use of a shorter screw) so as to not pass through the tridate cartilage. In that configuration the screw will not affect the growth plate and so bone growth on that side will not be modulated. Accordingly, by controlling screw depth in this manner, either by controlling placement of the screws or using screws of different lengths, unilateral bone growth modulation can be achieved.

Using present implant system 402, therefore, a minimally invasive, osteotomy-free, repositioning of the acetabulum using strategic pelvic growth modulation can be achieved. This is a paradigm changing treatment for acetabular dysplasia and is highly innovative over conventional approaches that require invasive osteotomy or thermal ablative procedures. As such, present implant system 402 can provide a beneficial alternative approach for the treatment of hip disorders, such as hip dysplasia by converting a large, open, surgical procedure that relies on soft tissue releases and boney cuts requiring extended, post-operative rehabilitation into a simple, nearly painless, and potentially outpatient procedure without substantial post-operative restrictions.

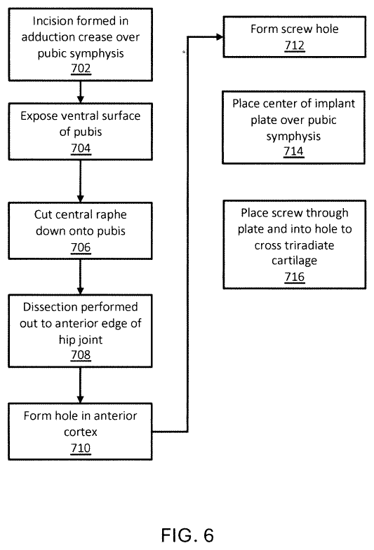

FIG. 6 is a flow chart depicting method 700 for installing the present implant device to modulate hip socket growth. At block 702, after preparing a patient according to normal procedure, a longitudinal incision, approximately 4 cm, is made in the adduction crease over the pubic symphysis. Blunt soft tissue dissection follows incision to separate the muscle from the midline raphe (e.g., by about 1 cm on each side) and the pubis can then palpated and muscle swept off the ventral surface of the pubis to, at block 704, expose the ventral surface of the pubis. Scissors or another cutting tool is then used to cut the central raphe down onto the pubis, and elevators used to tease the tissue off the pubic symphysis, at block 706. Dissection is then performed to expose the top of the pubis as well as the inferior margin allowing Homan type retractors to be placed into the right and left obturator foramen and over the top of the pubis. Additional dissection can be performed based on the style of implant used.

At block 708, dissection is performed along the superior and inferior margin of the superior rami was performed out to the anterior edge of the hip joint. At block 710, an awl or comparable tool is used to create a hole in the anterior cortex of the rami just medial to the superior aspect of the hip joint. At block 712, a screw hole is formed using, for example, a Lenke style spine awl, in a path for the screw medial to the hip joint, lateral and distal to the inner table and pelvic brim, that crossed the anterior limb of the triradiate cartilage. This can be done under fluoroscopy, with Steinmann pins being placed in these tracks and confirmed under fluoroscopy. At block 714, the center of the two-holed plate (an example plate may include Hinge plate Pega Medical/Orthopediatrics, Warsaw IN) is positioned over the symphysis bridging the symphysis anteriorly and at block 716 a canulated screw placed over the Steinmann pins crossing the triradiate cartilage under fluoroscopy.

Blocks 708 through 716 can then be repeated for a second screw into a second hip socket.

When performed method 700, incisions can be irrigated with cephazolin in sterile saline and allowed to soak for about four minutes. Then the incisions can be irrigated with sterile saline and closed. The fascia can be closed using 0-vicryl in a Ford interlocking suture pattern, 2-0 vicryl can be used to close the subcuticular layer (simple interrupted pattern). Skin can be stapled using stainless steel 35W staples. Incisions can be instilled with bupivacaine.

In various embodiments, the present disclosure provides for minimally invasive guided growth of the pelvis and acetabulum to gain hip stability and femoral coverage. The disclosure provides for guiding rotation of the sides of the pelvis using a hinge, while simultaneously inhibiting growth through the anterior limbs of the triradiate cartilage to change pelvic structure in a therapeutic manner. In doing so, the present disclosure provides a device in combination with a procedure that can correct acetabular dysplasia without cutting the pelvis bone, and without a prolonged recovery or limited weightbearing. Given the minimal invasive nature of the procedure, in many cases patients can immediately bear weight. In accordance with the present disclosure, the placement of a hinge plate over the symphysis pubis, placement of screws through the plate and across the bilateral anterior limb of the triradiate cartilage to affect growth.

In one embodiment, the present disclosure provides a device and technique that places a hinged plate over the anterior pubic symphysis and screws across the anterior limbs of the triradiate cartilage that simultaneously guides growth to improve the coverage of the femoral head in a minimally invasive fashion without pelvic osteotomy.

The preceding detailed description is merely illustrative in nature and is not intended to limit the embodiments of the subject matter or the application and uses of such embodiments.

As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, or detailed description.

It should be noted that many alternative or additional functional relationships or physical connections may be present in an embodiment of the subject matter. In addition, certain terminology may also be used herein for the purpose of reference only, and thus are not intended to be limiting, and the terms “first”, “second” and other such numerical terms referring to structures do not imply a sequence or order unless clearly indicated by the context.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed:

1. A method for modulating pelvic bone growth, comprising:
providing an implant system comprising a plate including a first screw hole and a second screw hole;
placing the plate over an anterior pubic symphysis of a pelvis;
screwing a first screw through the first screw hole of the plate and into the pelvis, wherein the first screw penetrates a first anterior limb of a first triradiate cartilage of a first acetabulum of the pelvis; and
screwing a second screw through the first screw hole of the plate and into the pelvis, wherein the second screw penetrates a second anterior limb of a second triradiate cartilage of a second acetabulum of the pelvis.

2. The method of claim 1, wherein the plate is flexible.

3. The method of claim 2, wherein the plate includes a biocompatible polymer.

4. The method of claim 2, wherein the plate includes a biocompatible resin.

5. The method of claim 2, wherein the plate includes a hinge between the first screw hole and the second screw hole.

6. The method of claim 1, wherein the plate includes polyetheretherketone.

7. A method for modulating pelvic bone growth, comprising:
providing an implant system comprising a plate including a first screw hole and a second screw hole;
positioning the plate proximate to a pubic symphysis of a pelvis;
screwing a first screw through the first screw hole of the plate and into the pelvis, wherein the first screw penetrates a first anterior limb of a first triradiate cartilage of a first acetabulum of the pelvis to modulate a growth of the first acetabulum; and
screwing a second screw through the first screw hole of the plate and into the pelvis.

8. The method of claim 7, wherein the pelvis includes a second acetabulum and neither the first screw nor the second screw modulate a growth of the second acetabulum.

9. The method of claim 7, wherein the plate is flexible.

10. The method of claim 9, wherein the plate includes a biocompatible polymer.

11. The method of claim 9, wherein the plate includes a biocompatible resin.

12. The method of claim 9, wherein the plate includes a hinge between the first screw hole and the second screw hole.

13. A method, comprising:
providing an implant system comprising a first screw hole; and
inserting a first screw through the first screw hole of the implant system and into a pelvis, wherein the first screw penetrates a first anterior limb of a first triradiate cartilage of a first acetabulum of the pelvis to modulate a growth of the first acetabulum.

14. The method of claim 13, wherein the implant system includes a second screw hole and the method further comprises inserting a second screw through the first screw hole of the implant system and into the pelvis.

15. The method of claim 14, wherein the pelvis includes a second acetabulum and the second screw does not penetrate a second anterior limb of a second triradiate cartilage of the second acetabulum.

16. The method of claim 15, wherein neither the first screw nor the second screw modulate a growth of the second acetabulum.

17. The method of claim 14, wherein the implant system includes a hinge between the first screw hole and the second screw hole.

18. The method of claim 13, wherein the implant system is flexible.

19. The method of claim 13, wherein the implant system includes a biocompatible polymer.

20. The method of claim 13, wherein the implant system includes a biocompatible resin.
